(19) United States
(12) Patent Application Publication
(10) Pub. No.: US 2026/0157760 A1
(43) Pub. Date: Jun. 11, 2026

(54) CUTTING GUIDE FOR FEMORAL NECK CUT

(71) Applicants: Smith & Nephew, Inc., Memphis, TN (US); Smith & Nephew Orthopaedics AG, Zug (CH)

(72) Inventors: Samuel C. Dumpe, Sewickley, PA (US); Branislav Jaramaz, Pittsburgh, PA (US); Eric Ashuckian, Pittsburgh, PA (US); Parker Hill, Pittsburgh, PA (US); Ran Schwarzkopf, New Rochelle, NY (US); Edward T. Davis, Worcestershire (GB)

(73) Assignees: Smith & Nephew, Inc., Memphis, TN (US); Smith & Nephew Orthopaedics AG, Zug (CH)

(21) Appl. No.: 19/269,520

(22) Filed: Jul. 15, 2025

Related U.S. Application Data
(60) Provisional application No. 63/676,676, filed on Jul. 29, 2024.

(51) Int. Cl. A61B 17/15; A61B 17/00

(52) U.S. Cl. CPC A61B 17/15; A61B 2017/00017

(57) ABSTRACT

A cutting guide is provided to assist a surgeon with resecting a portion of a patient's femoral neck or another bone. The cutting guide may include an opening configured to affix the cutting guide to at least one pin inserted into the patient's femoral neck, a proximal cutting surface, and a distal cutting surface. The proximal cutting surface defines a proximal cutting plane on the femoral neck and the distal cutting surface defines a distal cutting plane such that when cuts are made alone the proximal cutting plane and the distal cutting plane, a section of the femoral neck is removable. The pin may be inserted in the section of the femoral neck to be removed by an automated pin insertion tool. The tool may be configured to only operate when the pin is properly positioned and aligned to the femoral neck.

### CROSS-REFERENCE TO RELATED APPLICATIONS

This is a non-provisional of, and claims the benefit of the filing date of, U.S. provisional patent application number 63/676,676, filed Jul. 29, 2024, entitled “Cutting Guide For Femoral Neck Cut,” the entirety of which application is incorporated by reference herein.

### FIELD OF THE DISCLOSURE

The present disclosure relates generally to orthopedic devices and methods and more particularly to a cutting guide arranged and configured to resect a patient's bone. For example, the cutting guide may be used to resect a section of the femoral head and/or neck in a total hip arthroplasty.

### BACKGROUND

Many surgical procedures require preparation of a bone surface to receive an implant. In the case of a hip procedure, such as a total hip arthroplasty, a hip implant may be used to replace the natural head of the patient's femur. For example, a femoral implant may be implanted within the patient's femur. The femoral implant may include an intramedullary stem component, which is implanted into the intramedullary canal of the patient's femur and a hemispherical-shaped prosthetic head component, which is secured to the stem component and configured to interact with the patient's acetabulum or a hip implant (e.g., acetabulum cup), whichever the case may be. Similar implants may be used for other bones or joints requiring replacement, such as a humerus bone in a patient's shoulder.

In hip procedures, generally speaking, to resect the patient's femoral head, a cut is made to expose the hip joint including the upper femoral shaft and the femoral head. Next, a section of the femoral neck is resected, and the femoral head is removed. This allows access to the patient's acetabulum and femoral intramedullary canal to complete the subsequent steps of the procedure of acetabular reaming and femoral broaching. During resection of the femoral neck, a “napkin ring” section of the femoral neck may be removed. A single cut of the femoral neck may be insufficient because the femoral head requires space for the femoral head to be withdrawn from the socket (e.g., acetabulum). Thus, a disc or “napkin ring” of a defined height is removed such that the femoral head may be backed out of the socket (e.g., a “napkin ring” involves the formation of a proximal neck cut and a distal neck cut in order to create a section of bone, which can be removed to subsequently create space for removing the patient's femoral head from the patient's acetabulum).

However, the step of removing the femoral head is often done quickly and without great care. For example, some surgeons may eyeball an appropriate depth and freehand saw cut the patient's neck while attempting to remain perpendicular to the femoral neck axis. For example, some surgeons may mark the femoral neck using anatomical landmarks like the greater trochanter and lesser trochanter to define the inclination of the planar cut. Thereafter, using a cutting saw and the markings, the napkin ring section of the femoral neck may be resected using a free-hand technique. Attempts to cut and remove the femoral head may also be hampered via limited surgical site access and/or lack of surgeon experience.

Nevertheless, resection of a portion of the femoral head is a critical aspect of a hip procedure as it defines the placement of the subsequently implanted femoral implant. A poorly positioned femoral neck cut may negatively affect post-operative measures like leg length and offset, and thus negatively affect patient outcomes. This is particularly true as the use of collared femoral stems becomes more frequent.

Moreover, during certain total hip arthroplasty procedures, where the patient is placed in a supine position, such as a direct anterior (DA) approach, cutting the femoral neck may be rendered more challenging as access and visibility is limited due to the patient's supine position. Placing a patient in a supine position may restrict views of anatomical landmarks guiding neck cut position and trajectory.

It would be beneficial to provide a cutting guide that enables precise placement of the proximal cutting plane and the distal cutting plane. A device that provides guidance to the two cuts required would make the process easier and faster. Further, a device would be beneficial to provide a more accurate cut. When cut in a freehand fashion, the two cuts may not be in the proper plane, may not be at a proper location of the femoral neck, or may not be aligned with one another. Additionally, surgeons often encounter difficulties removing the napkin ring from the femoral neck after the cut due to compression of the bone sections and the limited access. It is with respect to these and other considerations that the present disclosure may be useful.

### BRIEF SUMMARY

This Summary is provided to introduce a selection of concepts in a simplified form that are further described below in the Detailed Description. This Summary is not intended to identify key features or essential features of the claimed subject matter, nor is it intended as an aid in determining the scope of the claimed subject matter.

A cutting guide is described herein that assists a surgeon with resecting a portion of a femoral neck or other bone. The cutting guide includes an opening configured to affix the cutting guide to at least one pin inserted into a bone, a proximal cutting surface, and a distal cutting surface. The proximal cutting surface defines a proximal cutting plane on the bone and the distal cutting surface defines a distal cutting plane such that when cuts are made along the proximal cutting plane and the distal cutting plane, a section of the bone is removable. The pin may be inserted in the section of the bone to be removed by an automated pin insertion tool. The tool may be configured to only operate when the pin is properly positioned and aligned to the bone.

In some examples, a cutting guide for resecting a portion of a bone, includes a body including one or more openings to receive one or more pins inserted into a bone, a proximal cutting surface, and a distal cutting surface. The proximal cutting surface defines a proximal cutting plane on the bone and the distal cutting surface defines a distal cutting plane such that when cuts are made along the proximal cutting plane and the distal cutting plane, a section of the bone is removable.

In any preceding or subsequent example, the one or more openings include first and second openings. In any preceding or subsequent example, one of the one or more openings is configured as a pin opening sized and configured to receive a first pin, the second opening is configured as an elongated channel sized and configured to receive a second pin. In any preceding or subsequent example, the first and second openings are configured as first and second separate and distinct channels sized and configured to receive first and second pins, respectively.

In any preceding or subsequent example, the pin is inserted into the bone in the section of the bone to be removed. In any preceding or subsequent example, the cutting guide may further include a spring-operated clamping mechanism to clamp the cutting guide to the pin when the cutting guide is affixed to the pin. In any preceding or subsequent example, one or more of the proximal cutting surface and the distal cutting surface may be located in a slot in a body of the cutting guide.

In any preceding or subsequent example, when the bone is cut along the proximal cutting plane and the distal cutting plane, a section of the bone is removable.

In any preceding or subsequent example, the bone is a femoral neck. In any preceding or subsequent example, the removable section of the femoral neck is of a thickness to allow removal of a femoral head from a socket of a hip. In any preceding or subsequent example, the bone is a humeral neck. In any preceding or subsequent example, the removable section of the humeral neck is of a thickness to allow removal of a humeral head from a socket of a shoulder. In any preceding or subsequent example, the cutting guide is manufactured of one or more of stainless steel or titanium.

In some examples, a method of using a cutting guide to resect a portion of a bone includes identifying a proximal cutting plane and a distal cutting plane in the bone positioning at least two pins through the bone in a section of the bone between the proximal cutting plane and the distal cutting plane, affixing a cutting guide to at least one of the pins and proximate to the bone, and cutting the bone along the proximal cutting plane and the distal cutting plane by positioning a cutting tool adjacent to the cutting guide.

In any preceding or subsequent example, the method further includes removing the section of the bone between the proximal cutting plane and the distal cutting plane. In any preceding or subsequent example, the cutting guide has a proximal cutting surface that is aligned with the proximal cutting plane and a distal cutting surface that is aligned with the distal cutting plane when the cutting guide is affixed to the pins.

In any preceding or subsequent example, the cutting guide is affixed to the pin via an opening on a body of the cutting tool. In any preceding or subsequent example, the pins are positioned with a pin insertion tool. In any preceding or subsequent example, the bone is one of a femoral neck or a humeral neck.

In any preceding or subsequent example, the cutting guide has a spring-operated clamping mechanism to clamp the cutting guide to the pin when the cutting guide is affixed to the pin. In any preceding or subsequent example, the distal cutting surface or the proximal cutting surface is located in a slot in a body of the cutting guide.

In any preceding or subsequent example, the pin insertion tool is controlled by a computer system such that the pin insertion tool does not operate if the pin is not in an alignment with a determined pin alignment plane. In any preceding or subsequent example, the computer system monitors a position of the pin insertion tool via one or more trackers affixed to the pin insertion tool.

Examples of the present disclosure provide numerous advantages. For example, the cutting guide provides multiple benefits to a surgeon to improve the resection of the section of the femoral neck or other bone. The cutting guide allows the surgeon to more clearly define the region of the napkin ring to be removed. In conventional systems, the cut is often performed freehand. The cutting guide causes the cut to be in the preferred location and on the preferred plane. The cutting guide further defines the depth of the napkin ring by clarifying and defining the distal and proximal cutting surfaces. The cutting guide is secured to the pin and thus the bone to allow the cut to be consistent and to resist drift in the cut. The cutting guide improves the removal of the napkin ring because the pins and the cutting guide are affixed to the bone and may be withdrawn with the bone section.

Further features and advantages of at least some of the examples of the present disclosure, as well as the structure and operation of various examples of the present disclosure, are described in detail below with reference to the accompanying drawings.

### BRIEF DESCRIPTION OF THE SEVERAL VIEWS OF THE DRAWINGS

By way of example, specific examples of the disclosed device will now be described, with reference to the accompanying drawings, in which:

FIG. 1 is a perspective view of a cutting guide in accordance with one or more features of the present disclosure, the cutting guide may be affixed to a patient's bone such as, for example, a patient's femoral neck to facilitate resection of a portion of the femoral neck or a humeral neck to facilitate resection of a portion of the humeral neck.

FIG. 2 is a perspective view of a pin insertion tool, which may be used to affix one or more pins into the patient's bone (e.g., femoral neck).

FIG. 3 is a perspective view illustrating first and second pins affixed into the patient's bone (e.g., femoral neck).

FIG. 4 is a perspective view of a proximal cutting plane, a distal cutting plane, and first and second pins.

FIG. 5 is a perspective view of a cutting guide affixed to first and second pins and aligned with the distal cutting plane and the proximal cutting plane.

FIG. 6 is a perspective view of an alternate example of a cutting guide affixed to first and second pins and aligned with the distal cutting plane and the proximal cutting plane.

FIG. 7 is a side view of an example pin insertion tool, which may be used in accordance with one or more features of the present disclosure.

FIG. 8 illustrates a user interface of an alignment display depicting a misaligned pin insertion.

FIG. 9 illustrates a user interface of an alignment display depicting an aligned pin insertion.

FIG. 10A is a side view of an alternate example of a cutting guide in accordance with one or more features of the present disclosure, the cutting guide including a spring button mechanism for locking the cutting guide to a pin. The spring button mechanism shown in a neutral position.

FIG. 10B is a side view of the cutting guide shown in FIG. 10A, the spring button mechanism shown in a locked position.

FIG. 11 illustrates an example method to resect a section of a patient's bone (e.g., femoral neck) using a cutting guide in accordance with one or more features of the present disclosure.

The drawings are not necessarily to scale. The drawings are merely representations, not intended to portray specific parameters of the disclosure. The drawings are intended to depict various examples of the disclosure, and therefore are not considered as limiting in scope. In the drawings, like numbering represents like elements.

### DETAILED DESCRIPTION

Various features or the like of a cutting guide, and corresponding method of use, will now be described more fully herein with reference to the accompanying drawings, in which one or more features of the cutting guide will be shown and described. It should be appreciated that the various features may be used independently of, or in combination, with each other. It will be appreciated that the cutting guide as disclosed herein may be embodied in many different forms and may selectively include one or more concepts, features, or functions described herein. As such, the cutting guide should not be construed as being limited to the specific examples set forth herein. Rather, these examples are provided so that this disclosure will convey certain features to those skilled in the art.

A cutting guide is described herein that assists a surgeon with resecting a portion of a patient's femoral neck. Although the present disclosure will describe and illustrate the cutting guide in accordance with resecting a portion of a patient's femoral neck, it should be appreciated that the cutting guide may be used in other areas. For example, the cutting guide may be used to resect a portion of a patient's humerus bone near a patient's shoulder joint, which has a similar neck and head configuration that may require a similar implant procedure. As such, the technology described herein may be used on any patient bone that may require a precise placement of a proximal cutting plane and/or a distal cutting plane. As a result, the present disclosure should not be limited to any particular surgical procedure or surgical area unless explicitly claimed.

The cutting guide includes an opening configured to affix the cutting guide to at least one pin inserted into a femoral neck, a proximal cutting surface, and a distal cutting surface. The proximal cutting surface defines a proximal cutting plane on the femoral neck and the distal cutting surface defines a distal cutting plane. The distal cutting guide being spaced from the proximal cutting guide such that when cuts are made along the proximal cutting plane and the distal cutting plane, a section of the femoral neck is removable. The pin may be inserted in the section of the femoral neck to be removed. In some examples, the pin may be inserted in the section of the femoral neck by an automated pin insertion tool. The pin insertion tool may be configured to only operate when the pin is properly positioned and aligned relative to the femoral neck.

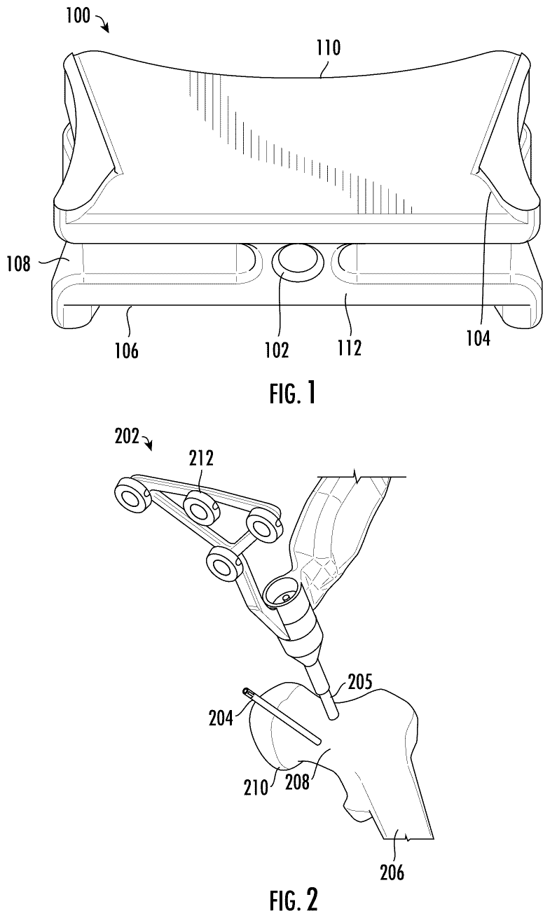

With reference to FIG. 1 and FIG. 2, an example of a cutting guide 100 configured to facilitate resection of a portion of a patient's femoral neck 208 is disclosed. The cutting guide 100 is configured to be affixed to the patient's femur via a first pin 204 and/or second pin 205 placed in the femoral neck 208. In some examples, the one or more pins may be referred to as simply pin 204 or collectively as pins 204, 204. The process for aligning and affixing the pins 204, 205 to the femoral neck 208 are described herein in greater detail.

As illustrated, the cutting guide 100 includes a proximal cutting surface 104 and a distal cutting surface 106. When affixed to the femoral neck 208, the proximal cutting surface 104 is aligned with a preferred proximal cutting plane 402 and the distal cutting surface 106 is aligned with a preferred distal cutting plane 404. The proximal cutting plane 402 and the distal cutting plane 404 may be established by a surgical team as described herein in greater detail.

In some examples, the cutting guide 100 includes a body 112 and a pin opening 102 positioned between the proximal cutting surface 104 and the distal cutting surface 106, the pin opening 102 arranged and configured to receive a first pin 204. The body 112 may also include one or more pin channels 108 between the proximal cutting surface 104 and the distal cutting surface 106. The pin channels 108 configured to receive one or more additional pins such as second pin 205 therein to secure the alignment of the cutting guide 100. In an alternate example, the cutting guide 100 may affix to a second pin 205 via a second pin opening 102. Incorporation of more or more pin channels 108 enables greater flexibility during positioning of the pins in contrast to incorporation of dedicated pin openings. The pins 204, 205 are placed in a napkin ring section of the femoral neck 208 between the proximal cutting plane 402 and the distal cutting plane 404. Placement in this section causes the pins 204, 205 to be removed when the napkin ring is removed from the femoral neck 208. Placement in the napkin ring is preferred because drilling holes for the pins 204, 205 in a section of bone may weaken the bone. In the examples, since the drilled section of bone is removed, the remaining femoral neck 208 is not compromised. In an alternate example, only a single pin is inserted in a pin opening 102 of the cutting guide 100. In an example, a bone may have characteristics that limit the number of pins 204, 205 that may be inserted.

The proximal cutting surface 104 provides a solid surface to allow a cutting tool of a surgeon to be secured in position. For example, the cutting tool may be pressed against the proximal cutting surface 104, rested against the proximal cutting surface 104, supported by the proximal cutting surface 104, or otherwise be provided with structure to allow a consistent, accurate, and efficient cutting process. Similarly, the distal cutting surface 106 provides a solid surface to allow a cutting tool of a surgeon to be secured in position to make the distal cut. In an alternative example, only a single cut is required (e.g., only a proximal cut or a distal cut may be required). A napkin ring removal may not be necessary based on a configuration of the bone, a socket, or other component. For example, a femoral head may be disposed such that removal from the socket does not require a napkin ring to be removed. In this instance, only a single cut on either the proximal cutting surface 104 or the distal cutting surface 106 is required.

The cutting guide 100 is illustrated with a semicircular contacting region 110. The contacting region 110 may be pressed against the femoral neck 208 when the cutting guide 100 is secured to the pins 204, 205. The semicircular configuration may allow the cutting guide 100 to conform to the shape of the semicircular region of the femoral neck 208.

The cutting guide 100 may be constructed of any suitable material. For example, the cutting guide 100 may be constructed of a durable metal, such as stainless steel or titanium. In another example, the cutting guide 100 may be constructed of a ceramic or one or more plastics, such as a thermoplastic or a thermoset.

A pin insertion tool 202 can include an attachment assembly configured to interchangeably engage a medical fastener and a cutting element or bone removal tool. The pin insertion tool 202 can include a drive assembly coupled to the attachment assembly. The attachment assembly can be configured to automatically release the medical fastener, such as a pin 204, in response to the drive assembly reaching its end or distal-most position to place the medical fastener at a predetermined depth within the bone.

The pin insertion tool 202 can be tracked by a camera that optically detects the trackers 212 and communicates that information to a computer system, such as a surgical computer, which is also tracking the bones within the surgical space. The computer system may compare the location of the pin insertion tool 202 with a pre-determined surgical plan.

In some examples, the pin insertion tool 202 is configured to activate or turn ON a drilling function when the pin insertion tool 202 is in a position where the surgical plan indicates the pin 204 should be placed and turns OFF when the pin insertion tool 202 is out of alignment. That is, the pin insertion tool 202 will not rotate and thereby not allow the surgeon to insert the pin 204, 205 if the pin 204, 205 is not at a proper location and is not in a proper alignment. The pin insertion tool 202 may stop a drill or other insertion mechanism when the pin 204, 205 is out of alignment. The pin insertion tool 202 may turn a drilling mechanism ON when the pin 204, 205 is back in proper alignment. The pin insertion tool 202 further may disengage or stop the drilling mechanism when the pin has reached a defined or configured depth.

The computer system can be configured to identify one or more trackers 212. The trackers may also be referred to as markers or fiducials. The computer system determines the pose (i.e., position and orientation) of the robotic arm controlling the pin insertion tool 202. In another example, the computer system tracks the pin insertion tool 202 via an electromagnetic tracking system instead of, or in addition to, a camera system.

As illustrated in FIG. 2, the pin insertion tool 202 is inserting a pin 205 into the femoral neck 208. A first pin 204 is already inserted. The femoral neck 208 is disposed between the femoral head 210 and the body of the femur 206.

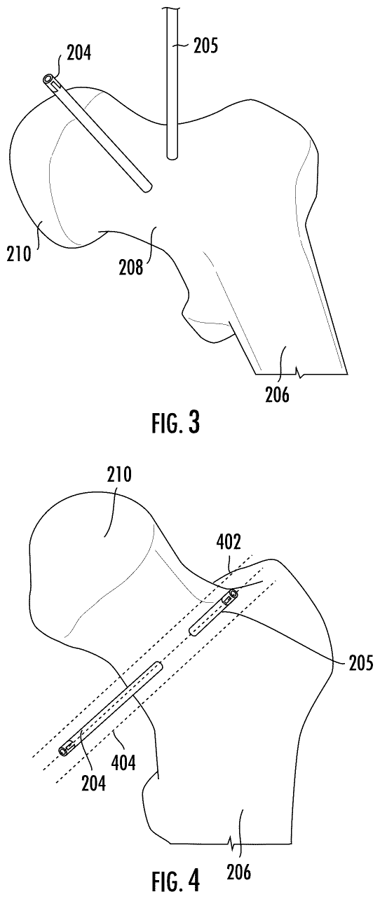

FIG. 3 is a perspective view of first pin 204 and second pin 205 affixed into a femoral neck 208. The pins 204, 205 are illustrated as protruding from the femoral neck 208 after insertion by the pin insertion tool 202. The pins 204, 205 are placed at a particular angle and position in the femoral neck 208 between the femoral head 210 and the shaft of the femur 206. The position and angle of the pins 204, 205 are configured to allow connection by the cutting guide 100 in a desired location.

Further, the pins 204, 205 may be used to assist with removing the napkin ring of bone. The pins 204, 205 may provide a useful handle or gripping tool for removing the bone.

FIG. 4 is a perspective view illustrating a proximal cutting plane 402, a distal cutting plane 404, and the first pin 204 and second pin 205.

As illustrated, the pins 204, 205 are inserted into the femoral neck 208 and protrude therefrom. As illustrated, the proximal and distal cutting planes 402, 404 define the edges of the napkin ring of bone that is removed in the process. The proximal cutting plane 402 and the distal cutting plane 404 may be determined during the planning stages of the procedure. The plan may use factors such as the type of implant being used, the size of the femur 206 and the femoral neck 208, the final implant position, and other factors to determine from where the napkin ring is to be removed and how thick the napkin ring should be. The size and position of the napkin ring determines the cutting planes for the proximal side and the distal side of the napkin ring.

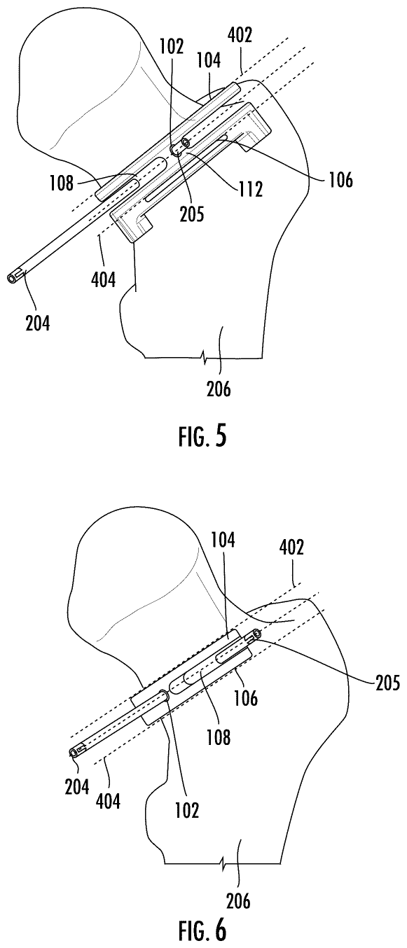

FIG. 5 is a perspective view of a cutting guide 100 affixed to pins 204, 205 and aligned with the distal cutting plane 404 and the proximal cutting plane 402.

The first pin 204 and second pin 205 are inserted in alignment with the proximal cutting plane 402 and the distal cutting plane 404 as described above. The cutting guide 100 is positioned with the second pin 205 being inserted through the pin opening 102. In addition, the first pin 204 is received within the pin channel 108. Thus, the cutting guide 100 is affixed to the femoral neck 208 via the first pin 204 and second pin 205. The cutting guide 100 is secured such that a surgeon may contact the cutting guide 100 with a cutting tool without dislodging and/or displacing (e.g., moving) the cutting guide 100.

The proximal cutting surface 104 of the cutting guide 100 is aligned with the proximal cutting plane 402. That is, when a surgeon places a cutting tool along the proximal cutting surface 104, the cut in the femoral neck 208 will align with the proximal cutting plane 402. Similarly, the distal cutting surface 106 is aligned with the distal cutting plane 404.

In some examples, the distal cutting surface 106 is illustrated as being a slot, channel, or opening in the body 112 of the cutting guide 100. The slot may serve to capture the blade of the cutting tool to ensure that the cut occurs at the proper location. For example, the blade is inserted into the slot and the slot prevents the blade from straying away from the distal cutting plane 404. In alternate examples, a slot is not used. In such a case, the surgeon uses the distal cutting surface 106 as a guide to support and direct the blade.

In another example, the distal cutting plane 404 may be configured to be the bottom of the cutting guide 100. Different cutting guide configurations may be used to define the proximal cutting surface 104 and the distal cutting surface 106 as long as the surfaces are aligned with the proximal cutting plane 402 and the distal cutting plane 404 as defined by the resection plan. For example, the cutting guides 100 may be provided in a kit including various sized cutting guides 100 having different heights as defined by the proximal and distal cutting surfaces 104, 106.

FIG. 6 is a perspective view of an alternate example of the cutting guide 100 affixed to pins 204, 205 and aligned with the distal cutting plane 404 and the proximal cutting plane 402. As illustrated, the cutting guide 100 includes a pin opening 102 and a single pin channel 108. The cutting guide 100 is positioned with the first pin 204 being inserted through the pin opening 102. In addition, the second pin 205 is received within the pin channel 108. Thus, the cutting guide 100 is affixed to the femoral neck 208 via the first pin 204 and second pin 205.

The cutting guide 100 including a distal cutting surface 106 that is at the bottom of the cutting guide 100 instead of in a slot or opening. The distal cutting surface 106 is aligned with the distal cutting plane 404. The proximal cutting surface 104 is at the top surface of the cutting guide 100 aligned with the proximal cutting plane 402.

In the alternate configuration, the cutting guide 100 is affixed to the pin 204 via the pin opening 102 in a similar manner as the cutting guide 100 of FIG. 5.

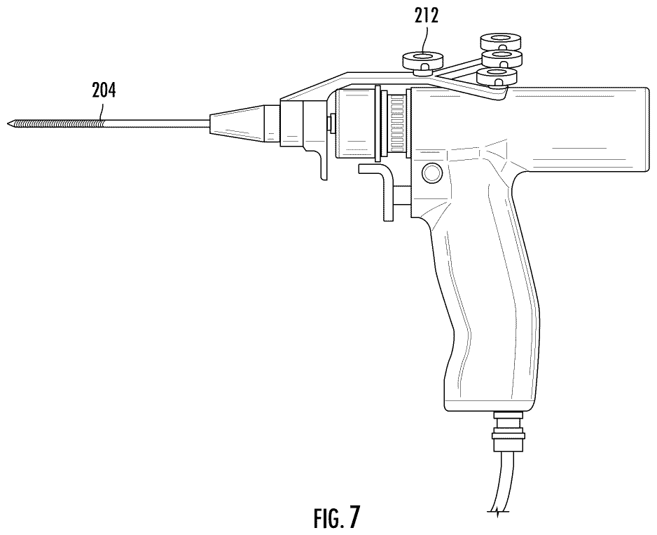

FIG. 7 is a side view of a pin insertion tool 202.

The pin insertion tool 202 pictured is the same as, or similar to, the pin insertion tool 202 described with respect to FIG. 2. The pin insertion tool 202 is illustrated with the pin 204 prepared for insertion into the femoral neck 208. The pin insertion tool 202 is illustrated with four trackers 212 for monitoring and directing the insertion location and angle of the pin 204. One example of a pin insertions tool 202 that installs medical fasteners at a predetermined depth is described in the PCT application published as WO2021163474A1 filed Feb. 12, 2021 and entitled “Systems for Robotic-Assisted Insertion of Medical Fasteners,” the entirety of which is incorporated herein by reference.

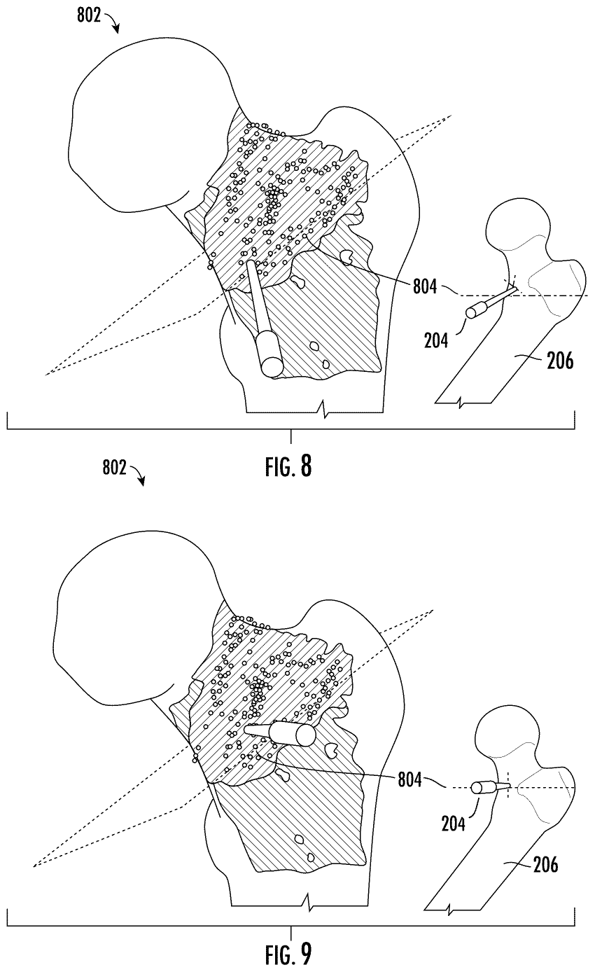

FIG. 8 illustrates a user interface of an alignment display 802 depicting a misaligned pin insertion.

In some examples, the pin insertion tool 202 is directed by a computer system. As described with respect to FIG. 2, the computer system monitors the trackers 212 and ensures the correct placement of the pins 204, 205. FIG. 8 illustrates a user interface that may be displayed to a surgeon or other user. The user interface has an alignment display 802 that displays a rendering of the femoral neck 208, femoral head 210, and the shaft of the femur 206. The alignment display 802 illustrates an angle of a pin alignment plane 804 that the surgeon has identified as the desired placement, angle, and pitch. In the left portion of the alignment display 802, the pin alignment plane 804 is illustrated by the dashed lines defining the borders of the plane 804. In the right portion of the alignment display 802, a side view of the pin alignment plane 804 is illustrated by the dashed line.

By monitoring the trackers 212, the computer system determines if the pin insertion tool 202 is aligned to the pin alignment plane 804. If the pin insertion tool 202 is not aligned, then the computer system may take an action, such as to stop the pin insertion tool 202 from operating. Only when the pin insertion tool 202 is aligned will the surgeon be able to insert the pin, such as pin 204.

In the example illustrated in FIG. 8, a misaligned pin 204 is shown. The pin alignment plane 804 is indicated in the rightmost inset image illustrating that the misaligned pin 204 is not at the proper location and is not at an angle consistent with the pin alignment plane 804. Further, a crosshair indicating a location of a tip of the misaligned pin 204 is indicated. The crosshair illustrates that the tip of the misaligned pin 204 is not in the pin alignment plane 804. The horizontal line illustrating the pin alignment plane 804 indicates that the angle of the misaligned pin 204 is not parallel with the pin alignment plane 804. In this case, the pin insertion tool 202 would not allow the surgeon to insert the pin 204. The display 802 may be color-coded to indicate that the location and/or angle is incorrect, such as by displaying the crosshair and/or the pin alignment plane 804 in red or yellow.

FIG. 9 illustrates a user interface of an alignment display 802 depicting an aligned pin insertion.

In the example illustrated in FIG. 9, an aligned pin 204 is shown. The pin alignment plane 804 is indicated in rightmost inset image illustrating that the aligned pin 204 is at the proper location and is at an angle consistent with the pin alignment plane 804. Further, a crosshair indicating a location of a tip of the aligned pin 204 is indicated. The crosshair illustrates that the tip of the aligned pin 204 is in the pin alignment plane 804. In this case, the pin insertion tool 202 would allow the surgeon to insert the pin 204. The display may be color-coded to indicate that the location and/or angle is correct, such as by displaying the crosshair and/or the pin alignment plane 804 in green.

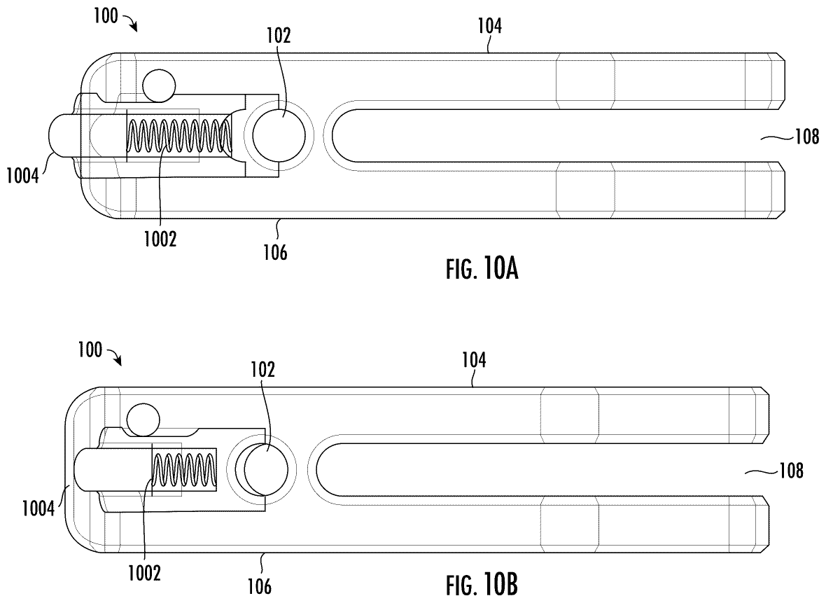

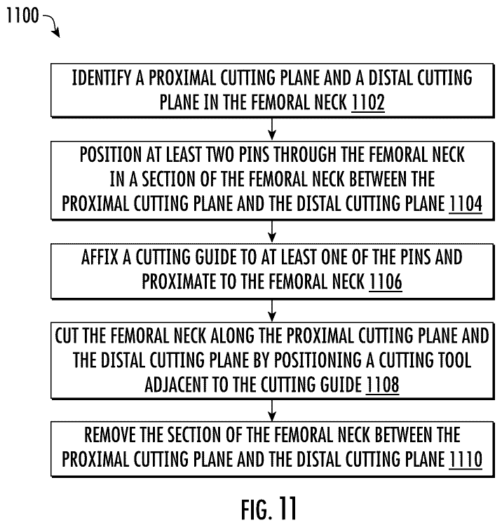

FIG. 10A is a side view of an alternate example of a cutting guide 100 including a spring-operated clamping mechanism for securing the cutting guide 100 to a pin 204. The spring-operated clamping mechanism illustrated in a neutral position.

The spring-operated clamping mechanism, and hence the cutting guide 100, may be configured with a locking spring 1002 for securing the cutting guide 100 to a pin, such as pin 204. The spring 1002 may be depressed with a spring button 1004 to cause the cutting guide 100 to grip the pin 204. The surgeon may use the gripped pin to assist with removal of the napkin ring of bone. For example, the surgeon may depress the spring 1002 to grip the pin 204, and concurrently grip the cutting guide 100 (and thus the pin 204), and withdraw the cutting guide 100, the pin 204, and the napkin ring of bone from the femoral neck 208.

This securing of the cutting guide 100 to the pin 204 may further prevent the cutting guide 100 from slipping from the pin 204, spinning around the femoral neck 208, vibrating while the cutting tool is operating, or otherwise becoming insecure. The spring 1002 may be configured with a locking spring button 1004.

As illustrated in FIG. 10A, the spring button 1004 is not depressed and the spring 1002 is in a neutral position. The pin opening 102 is at a full open position to allow a pin 204 to be inserted. The pin opening clamp 1006 does not cover any portion of the pin openings 102.

FIG. 10B is a side view of a cutting guide 100 with a spring 1002 for locking the cutting guide 100 to a pin 204. The spring 1002 is in a locked position.

As illustrated in FIG. 10A, the spring button 1004 is depressed and the spring 1002 is in a compressed position. The pin opening 102 is at reduced position to allow a pin 204 to be clamped into position. The pin opening clamp 1006 does cover at least a portion of the pin openings 102. When the pin 204 is clamped by the pin opening clamp 1006, the spring button locks into position and the spring 1002 forces the pin opening clamp 1006 to put frictional pressure on the pin 204.

FIG. 11 illustrates a method 1100 to resect a section of a femoral neck 208 using a cutting guide 100. The blocks of the method 1100 are described herein as being performed by a surgeon, but each function of the method 1100 may alternatively be performed by a surgeon, an operator, a technician, or an automated process.

In block 1102, a surgeon identifies a proximal cutting plane 402 and a distal cutting plane 404 in the femoral neck 208. The proper position, angle, attitude, or other location of the proximal cutting plane 402 and the distal cutting plane 404 may be determined by a surgeon, by a three-dimensional mapping of the femoral neck 208, by an automated process, or by any other suitable method. The proximal cutting plane 402 and the distal cutting plane 404 may be marked or otherwise denoted on the femoral neck 208 or on a user interface of an automated process.

In block 1104, the surgeon positions at least two pins 204, 205 through the femoral neck 208 in a section of the femoral neck 208 between the proximal cutting plane 402 and the distal cutting plane 404. The pins 204, 205 may be positioned in any suitable method described herein. For example, the pin insertion tool 202 may be used to properly align and install the pins 204, 205.

In block 1106, the surgeon affixes a cutting guide 100 to at least one of the pins 204, 205 and proximate to the femoral neck 208. The cutting guide 100 may be affixed to one or more of the pins 204, 205 as described herein.

In block 1108, the surgeon cuts the femoral neck 208 along the proximal cutting plane 402 and the distal cutting plane 404 by positioning a cutting tool adjacent to the cutting guide 100.

In block 1110, the surgeon removes the section of the femoral neck 208 between the proximal cutting plane 402 and the distal cutting plane 404.

While the present disclosure refers to certain examples, numerous modifications, alterations, and changes to the described examples are possible without departing from the sphere and scope of the present disclosure, as defined in the appended claim(s). Accordingly, it is intended that the present disclosure not be limited to the described examples, but that it has the full scope defined by the language of the following claims, and equivalents thereof. The discussion of any example is meant only to be explanatory and is not intended to suggest that the scope of the disclosure, including the claims, is limited to these examples. In other words, while illustrative examples of the disclosure have been described in detail herein, it is to be understood that the inventive concepts may be otherwise variously embodied and employed, and that the appended claims are intended to be construed to include such variations, except as limited by the prior art.

The foregoing discussion has been presented for purposes of illustration and description and is not intended to limit the disclosure to the form or forms disclosed herein. For example, various features of the disclosure are grouped together in one or more examples or configurations for the purpose of streamlining the disclosure. However, it should be understood that various features of the certain examples or configurations of the disclosure may be combined in alternate examples, or configurations. Any example or feature of any section, portion, or any other component shown or particularly described in relation to various examples of similar sections, portions, or components herein may be interchangeably applied to any other similar example or feature shown or described herein. Additionally, components with the same name may be the same or different, and one of ordinary skill in the art would understand each component could be modified in a similar fashion or substituted to perform the same function.

Moreover, the following claims are hereby incorporated into this Detailed Description by this reference, with each claim standing on its own as a separate example of the present disclosure.

As used herein, an element or step recited in the singular and proceeded with the word “a” or “an” should be understood as not excluding plural elements or steps, unless such exclusion is explicitly recited. Furthermore, references to “one example” of the present disclosure are not intended to be interpreted as excluding the existence of additional examples that also incorporate the recited features.

The phrases “at least one,” “one or more,” and “and/or,” as used herein, are open-ended expressions that are both conjunctive and disjunctive in operation. The terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, and counterclockwise) are only used for identification purposes to aid the reader's understanding of the present disclosure, and do not create limitations, particularly as to the position, orientation, or use of this disclosure. Connection references (e.g., engaged, attached, coupled, connected, and joined) are to be construed broadly and may include intermediate members between a collection of elements and relative to movement between elements unless otherwise indicated. As such, connection references do not necessarily infer that two elements are directly connected and in fixed relation to each other. All rotational references describe relative movement between the various elements. Identification references (e.g., primary, secondary, first, second, third, fourth, etc.) are not intended to connote importance or priority but are used to distinguish one feature from another. The drawings are for purposes of illustration only and the dimensions, positions, order and relative to sizes reflected in the drawings attached hereto may vary.

What is claimed is:

1. A cutting guide for resecting a portion of a bone, comprising:
a body including one or more openings to receive one or more pins inserted into a bone;
a proximal cutting surface; and
a distal cutting surface,
wherein the proximal cutting surface defines a proximal cutting plane on the bone and the distal cutting surface defines a distal cutting plane on the bone such that when proximal and distal cuts are made along the proximal cutting plane and the distal cutting plane, a section of the bone is removable.

2. The cutting guide of claim 1, wherein the one or more openings include first and second openings.

3. The cutting guide of claim 2, wherein one of the one or more openings is configured as a pin opening sized and configured to receive a first pin, the second opening is configured as an elongated channel sized and configured to receive a second pin.

4. The cutting guide of claim 2, wherein the first and second openings are configured as first and second separate and distinct channels sized and configured to receive first and second pins, respectively.

5. The cutting guide of claim 1, wherein the one or more pins are inserted into the bone in the section of the bone to be removed.

6. The cutting guide of claim 1, further comprising a spring-operated clamping mechanism to clamp the cutting guide to the pin when the cutting guide is affixed to the pin.

7. The cutting guide of claim 1, wherein one or more of the proximal cutting surface and the distal cutting surface are located in a slot in the body of the cutting guide.

8. The cutting guide of claim 1, wherein when the bone is cut along the proximal cutting plane and the distal cutting plane, a section of the bone is removable.

9. The cutting guide of claim 8, wherein the bone is a femoral neck.

10. The cutting guide of claim 9, wherein the removable section of the femoral neck is of a thickness to allow removal of a femoral head from a socket of a hip.

11. The cutting guide of claim 8, wherein the bone is a humeral neck.

12. The cutting guide of claim 11, wherein the removable section of the humeral neck is of a thickness to allow removal of a humeral head from a socket of a shoulder.

13. The cutting guide of claim 1, wherein the cutting guide is manufactured of one or more of stainless steel or titanium.

14. A method to use a cutting guide to resect a portion of a bone, comprising:
identifying a proximal cutting plane and a distal cutting plane in the bone;
positioning at least two pins through the bone in a section of the bone between the proximal cutting plane and the distal cutting plane;
affixing a cutting guide to at least one of the pins and proximate to the bone; and
cutting the bone along the proximal cutting plane and the distal cutting plane by positioning a cutting tool adjacent to the cutting guide.

15. The method of claim 14, further comprising:
removing the section of the bone between the proximal cutting plane and the distal cutting plane.

16. The method of claim 14, wherein the cutting guide has a proximal cutting surface that is aligned with the proximal cutting plane and a distal cutting surface that is aligned with the distal cutting plane when the cutting guide is affixed to the pins.

17. The method of claim 14, further comprising affixing the cutting guide to the pin via an opening on a body of the cutting tool.

18. The method of claim 14, wherein the pins are positioned with a pin insertion tool.

19. The method of claim 18, wherein the pin insertion tool is controlled by a computer system such that the pin insertion tool does not operate if the pin is not in alignment with a determined pin alignment plane.

20. The method of claim 14, wherein the bone is one of a femoral neck or a humeral neck.
